(19) United States
(12) Patent Application Publication
(10) Pub. No.: US 2022/0031164 A1
(43) Pub. Date: Feb. 3, 2022

(54) MRI-CT SYSTEM AND METHODS FOR SEQUENTIALLY IMAGING

(71) Applicant: SYNAPTIVE MEDICAL INC., Toronto (CA)

(72) Inventors: Alexander Gyles PANTHER, Toronto (CA); Geron Andrè BINDSEIL, London (CA)

(73) Assignee: SYNAPTIVE MEDICAL INC., Toronto (CA)

(21) Appl. No.: 17/443,964

(22) Filed: Jul. 29, 2021

Related U.S. Application Data
(63) Continuation of application No. 63/057,979, filed on Jul. 29, 2020.

(51) Int. Cl. A61B 5/00; A61B 5/055; G01R 33/421

(52) U.S. Cl. CPC A61B 5/0035; G01R 33/4215; A61B 5/0042; A61B 5/055

(57) ABSTRACT

An MRI-CT system and methods for sequentially (or simultaneously) imaging a subject involving a CT component for initially performing CT imaging, an MR component for subsequently performing MR imaging, the MR component and the CT component disposable in relation to one another in at least one of linearly aligned and colinearly aligned, and a movable barrier disposable between the CT component and the MR component, the movable barrier comprising a magnetic shield, and the movable barrier disposable in one of an open position and a closed position during MRI scanning by the MR component and in a closed position during CT scanning by the CT component.

### CROSS-REFERENCE TO RELATED APPLICATION(S)

This document is a nonprovisional patent application claiming the benefit of, and priority to, U.S. Provisional Patent Application No. 63/057,979, entitled “Combined MRI-CT within the same room separated by removable magnetic shielding barrier and connected with a co-axial patient transport system for sequential imaging,” and filed on Jul. 29, 2020, hereby incorporated by reference in its entirety.

### FIELD

The subject matter of the present disclosure generally relates to magnetic resonance imaging technology and computed tomography technology.

### BACKGROUND

In the related art, a magnetic resonance imaging—positron emission tomography (MRI-PET) imaging systems and a positron emission tomography—computerized tomography (PET-CT) system are used in pre-clinical environments. An after-market patient bed or gurney that is configured for use with these particular combined systems is typically used, wherein the patient is moved from one scanner to the other scanner, typically requiring a very large room for performing the scanning in order to avoid adverse electromagnetic radiation ramifications on the respective components. These related art MRI-PET and PET-CT systems are configured to accommodate small animals in a veterinary clinical environment and do not accommodate a human patient.

Also in the related art, another imaging approach involves a simultaneously scanning CT-MRI system, wherein combining the two systems necessitates compromising performance of one or both of the CT scanner and the MRI scanner. These related art systems use low-field permanent MRI systems, or traditional superconducting MRI systems having gaps, wherein such systems are technically challenging to engineer and expensive to manufacture. For a related art PET/CT and MR system, such systems are not disposed in the same room; and the system components are not linearly or co-linearly disposable. Although such related art systems may use a magnetic shielding disposed in the wall which separates the scanners, such related art shielding still requires that the scanners are disposed far from one another in a room.

However, a related art MRI scanner and a related art CT scanner have respective limitations in a clinical setting, such as involving separate, large, heavy, bulky, stationary, or immobile equipment as well as practical limitations and logistic limitations in moving a patient between MRI equipment and CT equipment. Tremendous risk to the patient is inherent in both moving the patient from a CT scanner to an MRI scanner as such equipment are typically located in different areas of a hospital) and in timely performing an MRI scan after performing a CT scan. Further, a related art CT scanner has a limit of 1 Gauss for magnetic field; therefore, the related art CT scanner must be disposed far from an MRI scanner. Even further, related art CT tables typically contain carbon fiber which is conductive. As such, related art CT tables are incompatible with MR scanning, thereby necessitating moving the patient from a CT table to an MR table in order to perform both types of imaging, wherein moving the patient introduces further risk to the patient. Therefore, a needs exists in the related art for an efficient manner of rapidly providing both MRI scanning and CT scanning, minimizing further trauma, to a patient, and overcoming the magnetic field limitations of related art CT scanners and tables.

### SUMMARY

To address at least the challenges in the related art, the subject matter of the present disclosure involves an MRI-CT systems and methods for sequential imaging, e.g., rapid sequential imaging. For example, a patient experiencing trauma, such as a head trauma, a stroke, a brain trauma, and the like, would initially undergo a CT scan which would indicate conditions, such as a skull fracture and internal bleeding, but the patient would also benefit from subsequently undergoing an MRI scan which would provide further information relating to a condition of soft tissue. For at least the foregoing reasons, the MRI-CT system and methods involve rapid sequential imaging, e.g., obtaining an MRI scan as quickly as possible after obtaining a CT scan, without moving the patient any significant distance in relation to an MRI imaging apparatus, a CT imaging apparatus, and a transporter, thereby avoiding further trauma to the patient, thereby reducing image acquisition time, and thereby reducing diagnostic time.

Generally, the MRI-CT system and the methods involve sequentially imaging a patient by initially performing a CT scan and immediately performing an MRI scan by using one piece of equipment located in one area. Further, the MRI-CT system and the methods involve a CT component in combination with a magnetic resonance (MR) component, the MR component having a small fringe field and a removable barrier or door comprising a magnetic shield, and the CT component and the MR component disposed in relation to one another in at least one of linearly aligned and colinearly aligned. Even further, the MRI-CT system and the methods involve a transporter configured to facilitate seamless movement of a patient from the CT component to the MR component. The MRI-CT system and the methods also involve disposing the CT component and the MR components much closer together and requiring less floor space than do the related art systems and methods.

In accordance with an embodiment of the present disclosure, an MRI-CT system for sequentially imaging a subject comprises: a CT component for initially performing CT imaging; an MR component for subsequently performing MR imaging, the MR component and the CT component disposable in relation to one another in at least one of linearly aligned and colinearly aligned; and a movable barrier disposable between the CT component and the MR component, the movable barrier comprising a magnetic shield, and the movable barrier disposable in one of an open position and a closed position during MRI scanning by the MR component and in a closed position during CT scanning by the CT component

In accordance with an embodiment of the present disclosure, a method of providing an MRI-CT system for sequentially imaging a subject comprises: providing a CT component for initially performing CT imaging; providing an MR component for subsequently performing MR imaging, the MR component and the CT component disposable in relation to one another in at least one of linearly aligned and colinearly aligned; and providing a movable barrier disposable between the CT component and the MR component, providing the movable barrier comprising providing a magnetic shield, and providing the movable barrier comprising providing the movable barrier as disposable in one of an open position and a closed position during MRI scanning by the MR component and in a closed position during CT scanning by the CT component.

In accordance with an embodiment of the present disclosure, a method of sequentially imaging a subject, by way of an MRI-CT system, comprises: providing the MRI-CT system, providing the MRI-CT system comprising: providing a CT component for initially performing CT imaging; providing an MR component for subsequently performing MR imaging, the MR component and the CT component disposable in relation to one another in at least one of linearly aligned and colinearly aligned; and providing a movable barrier disposable between the CT component and the MR component, providing the movable barrier comprising providing a magnetic shield, and providing the movable barrier comprising providing the movable barrier as disposable in one of an open position and a closed position during MRI scanning by the MR component and in a closed position during CT scanning by the CT component; providing a bridge having at least one portion, providing the bridge comprising configuring the bridge to engage a transporter, and providing the bridge comprising configuring the bridge as disposable between the CT component and the MR component; providing the transporter, providing the transporter comprising configuring the transporter to engage the bridge, wherein at least one of providing the bridge and providing the transporter comprises providing a moving feature to facilitate moving the transporter forward in a sequence and backward from the sequence; disposing the subject on the transporter; engaging the transporter with the bridge; moving the transporter in relation to the CT component by using the bridge; performing CT imaging of the subject by using the CT component; moving the transporter in relation to the MR component by using the bridge; and performing MR imaging of the subject by using the MR component.

Some of the features in the present disclosure are broadly outlined in order that the section entitled Detailed Description is better understood and that the present contribution to the art may be better appreciated. Additional features of the present disclosure are described hereinafter. In this respect, understood is that the present disclosure is not limited in its application to the details of the components or steps set forth herein or as illustrated in the several figures of the being carried out in various ways. Also, understood is that the phraseology and terminology employed herein are for the purpose of the description and should not be regarded as limiting.

### BRIEF DESCRIPTION OF THE DRAWING(S)

The above, and other, aspects, features, and advantages of several embodiments of the present disclosure are described in the following Detailed Description as presented in conjunction with the several figures of the Drawing as follows.

FIG. 1 is a diagram illustrating a combined radiation therapy and MRI system comprising a radiation therapy machine and a magnetic resonance imaging (MRI) scanner, in accordance with an embodiment of the present disclosure.

FIG. 2A is a diagram illustrating a patient table in a first position operable in a combined radiation therapy and MRI system, in accordance with an embodiment of the present disclosure.

FIG. 2B is a diagram illustrating a patient table in a second position operable in a combined radiation therapy and MRI system, in accordance with an embodiment of the present disclosure.

FIG. 2C is a diagram illustrating a patient table in a third position operable in a combined radiation therapy and MRI system, as shown in FIG. 2C, in accordance with an embodiment of the present disclosure.

FIG. 2D is a diagram illustrating a patient table in a fourth position operable in a combined radiation therapy and MRI system, as shown in FIG. 2B, in accordance with an embodiment of the present disclosure.

FIG. 3A is a diagram illustrating a magnetic field intensity of the magnetic field produced by an MRI scanner of a combined radiation therapy and MRI system being used for imaging (in an imaging mode), in accordance with an embodiment of the present disclosure.

FIG. 3B is a diagram illustrating a magnetic field intensity of the magnetic field produced by a radiation therapy machine of a combined radiation therapy and MRI system being used for treating a patient (in a treatment mode), in accordance with an embodiment of the present disclosure.

FIG. 4A is a graph of a magnetic field of an MRI scanner having a 0.5-Tesla magnet, in accordance with an embodiment of the present disclosure.

FIG. 4B is a graph of a magnetic field of an MRI scanner having a 0.1665-Tesla magnet, in accordance with an embodiment of the present disclosure.

FIG. 5A is a diagram illustrating MRI guided radiation therapy of the combined radiation therapy and MRI system for imaging a head region by using an external radiation beam, in accordance with an embodiment of the present disclosure.

FIG. 5B is a diagram illustrating MRI guided radiation therapy for targeting the same head region, as shown in FIG. 5A, by using the external radiation beam, in accordance with an embodiment of the present disclosure.

FIG. 6A is a schematic diagram illustrating a top view of an MRI-CT system for sequentially imaging, in accordance with an embodiment of the present disclosure.

FIG. 6B is a schematic diagram illustrating a top view, an MRI-CT system for sequentially imaging, in accordance with an alternative embodiment of the present disclosure.

FIG. 7 is a schematic illustrating a top view of an MRI-CT system for sequentially imaging, operating in a CT imaging mode, in accordance with an embodiment of the present disclosure.

FIG. 8 is a schematic diagram illustrating a top view of an MRI-CT system for sequentially imaging, operating in an MRI imaging mode, in accordance with an embodiment of the present disclosure.

FIGS. 9A and 9B are schematic diagrams illustrating a perspective view of the transporter, engaged with the first portion of the bridge and the second portion of the bridge, for moving the patient through the CT component of an MRI-CT system S for sequentially imaging, as shown in FIGS. 6 and 7, in accordance with an embodiment of the present disclosure.

FIG. 10 is a schematic diagram illustrating a perspective view of the transporter, engaged with the second portion of the bridge, for moving the patient (not shown) to, and into, the MR component of an MRI-CT system S for sequentially imaging, as shown in FIGS. 7 and 8, in accordance with an embodiment of the present disclosure.

FIG. 11 is a flow diagram illustrating a method of providing an MRI-CT system for sequentially imaging, in accordance with an embodiment of the present disclosure.

FIG. 12 is a flow diagram illustrating a method of sequentially imaging by way of an MRI-CT system, in accordance with an embodiment of the present disclosure.

FIG. 13 is a schematic diagram illustrating a top view of an MRI-CT system for simultaneously imaging, in accordance with an alternative embodiment of the present disclosure.

Corresponding reference numerals or characters indicate corresponding components throughout the several figures of the Drawing. Elements in the several figures are illustrated for simplicity and clarity and have not necessarily been drawn to scale. For example, the dimensions of some of the elements in the figures may be emphasized relative to other elements for facilitating understanding of the various presently disclosed embodiments. Also, common, but well-understood, elements that are useful or necessary in a commercially feasible embodiment of the present disclosure are often not depicted in order to facilitate a less obstructed view of these various embodiments.

### DETAILED DESCRIPTION

As used herein, the terms “comprises” and “comprising” are to be construed as being inclusive and open ended, and not exclusive. Specifically, when used in the specification and claims, the terms “comprises” and “comprising” and variations thereof mean the specified features, steps or components are included. These terms are not to be interpreted to exclude the presence of other features, steps or components.

As used herein, the term “exemplary” means “serving as an example, instance, or illustration,” and should not be construed as preferred or advantageous over other configurations disclosed herein.

As used herein, the terms “about” and “approximately” are meant to cover variations that may exist in the upper and lower limits of the ranges of values, such as variations in properties, parameters, and dimensions. In one non-limiting example, the terms “about” and “approximately” mean plus or minus 10 percent or less.

Radiation oncologists generally prefer to use magnetic resonance-guidance for radiation therapy systems. However, the high magnetic fields of magnetic resonance imaging (MRI) systems can disturb the operation of the external beam generators, e.g., linear accelerators, of the radiation therapy system. In some implementations, the magnetic field of the MRI magnet is reduced or turned off when the radiation therapy is initiated so that the disturbance caused by the magnet to the radiation therapy system may be non-existent during radiation therapy. In this case, the MRI system can be located near the radiation therapy machine, which may allow the human subject to be expeditiously transported from the MRI scanner to the radiation therapy system on the same platform in the same room. This may allow imaging to be performed immediately before the radiation therapy, thereby yielding up-to-date MRI images of the human subject at the region of interest.

In one aspect, some implementations provide a method that includes: placing a human subject on a movable platform, wherein the movable platform is located in a room with a magnetic resonance imaging (MRI) scanner and a radiation therapy machine, the radiation therapy machine including an external beam generator and the MRI scanner including at least one magnet that generates a magnetic field; moving the platform into a first position such that the human subject is positioned to be imaged by the magnetic resonance imaging (MRI) scanner, the first position resulting in at least a portion of the human subject being located inside the magnet of the MRI scanner; operating the MRI scanner while the platform is in the first position to obtain an image of the human subject; moving the platform into a second position such that the human subject is position to receive radiation therapy from the radiation therapy machine; reducing the magnetic field generated by the magnet such that the magnetic field at the radiation therapy machine is below a threshold value; and while the platform is in the second position and the magnetic field at the radiation therapy machine is below the threshold value, operating the radiation therapy machine to perform radiation therapy on the human subject.

Implementations may include one or more of the following features. Reducing the magnetic field of the magnet may include turning off the magnet such that the magnetic field generated by the magnet at the radiation therapy machine is substantially zero during the radiation therapy. The magnet and the radiation therapy machine may be separated by less than 5 m. The magnetic field generated by the magnet is at least 0.1 Tesla to obtain the image of the human subject. The threshold value is no more than 1 Gauss. The threshold value is no more than 60% of a strength of the magnetic field during magnetic resonance imaging at the radiation therapy machine.

The method may additionally include reducing the magnetic field generated by the magnet while moving the platform into the second position such that the human subject is positioned to receive the radiation therapy from the radiation therapy machine. The method may further include ramping up the magnetic field generated by the magnet at completion of the radiation therapy.

The method may further include: moving the platform from the first position into the second position while fixing a region of interest of the human subject relative to the platform. The region of interest may include a head region. Operating the radiation therapy machine may include operating an external beam generator and a collimator to generate a radiation beam targeting at the region of interest. Operating the radiation therapy machine to perform radiation therapy may include operating the radiation therapy machine to perform external radiation beam therapy. Operating the radiation therapy machine to perform radiation therapy may further include using a fluoroscopic device to obtain fluoroscopic images of the human subject at the region of interest. Operating the radiation therapy machine to perform radiation therapy may further include fusing the MRI image with the fluoroscopic images to facilitate targeting the radiation beam at the region of interest. Fusing the MRI image on the fluoroscopic images includes aligning the MRI image with the fluoroscopic images by virtue of fixing the region of interest with respect to the platform. Fusing the MRI image on the fluoroscopic images may include using fiducial markers that are visible in both MRI and fluoroscopy. Operating the MRI scanner to obtain the image of the human subject may include operating the MRI scanner to obtain a 3-dimensional image of the human subject at the region of interest. Operating the MRI scanner to obtain the image of the human subject may include operating the MRI scanner to obtain a multi-slice image of the human subject at the region of interest.

In another aspect, some implementations provide a computer-assisted method that includes: placing a human subject on a movable platform, wherein the movable platform is located in a room with a magnetic resonance imaging (MRI) scanner and a radiation therapy machine, the radiation therapy machine including an external beam generator and the MRI scanner including at least one magnet that generates a magnetic field; moving the platform into a first position such that the human subject is positioned to be imaged by the magnetic resonance imaging (MRI) scanner, the first position resulting in at least a portion of the human subject being located inside the magnet of the MRI scanner; operating the MRI scanner while the platform is in the first position to obtain an image of the human subject; moving the platform into a second position such that the human subject is position to receive radiation therapy from the radiation therapy machine; reducing the magnetic field generated by the magnet such that the magnetic field at the radiation therapy machine is below a threshold value; and while the platform is in the second position and the magnetic field at the radiation therapy machine is below the threshold value, operating the radiation therapy machine to perform radiation therapy on the human subject.

Implementations may include one or more of the following features. Reducing the magnetic field of the magnet may include turning off the magnet such that the magnetic field generated by the magnet at the radiation therapy machine is substantially zero during the radiation therapy. The magnet and the radiation therapy machine may be separated by less than 5 m. The magnetic field generated by the magnet is at least 0.1 Tesla to obtain the image of the human subject. The threshold value is no more than 1 Gauss. The threshold value is no more than 60% of a strength of the magnetic field during magnetic resonance imaging at the radiation therapy machine.

The method may additionally include reducing the magnetic field generated by the magnet while moving the platform into the second position such that the human subject is positioned to receive the radiation therapy from the radiation therapy machine. The method may further include ramping up the magnetic field generated by the magnet at completion of the radiation therapy.

The method may further include moving the platform from the first position into the second position while fixing a region of interest of the human subject relative to the platform. The region of interest may include a head region. Operating the radiation therapy machine may include operating an external beam generator and a collimator to generate a radiation beam targeting at the region of interest. Operating the radiation therapy machine to perform radiation therapy may include operating the radiation therapy machine to perform external radiation beam therapy. Operating the radiation therapy machine to perform radiation therapy may further include using a fluoroscopic device to obtain fluoroscopic images of the human subject at the region of interest. Operating the radiation therapy machine to perform radiation therapy may further include fusing the MRI image with the fluoroscopic images to facilitate targeting the radiation beam at the region of interest. Fusing the MRI image on the fluoroscopic images includes aligning the MRI image with the fluoroscopic images by virtue of fixing the region of interest with respect to the platform. Fusing the MRI image on the fluoroscopic images may include using fiducial markers that are visible in both MRI and fluoroscopy. Operating the MRI scanner to obtain the image of the human subject may include operating the MRI scanner to obtain a 3-dimensional image of the human subject at the region of interest. Operating the MRI scanner to obtain the image of the human subject may include operating the MRI scanner to obtain a multi-slice image of the human subject at the region of interest.

In yet another aspect, some implementations provide an magnetic resonance imaging (MRI) guided radiation therapy system, the system including: an magnetic resonance imaging (MRI) scanner including a magnet configured to generate a magnetic field; a radiation therapy machine located within 5 m of the MRI scanner; a platform configured to transport a human subject from a first position in which at least a portion of the human subject is disposed in the magnet to obtain an magnetic resonance image thereof to a second position in which the portion of the human subject is disposed in the radiation therapy machine to receive a radiation therapy while the magnetic field at the radiation therapy machine has been reduced below a threshold value.

Implementations may include one or more of the following features. The magnet may be configured to generate a magnetic field at least 0.1 Tesla to obtain the image of the portion of the human subject. The threshold value may be no more than 1 Gauss. The threshold value may be no more than 60% of a strength of the magnetic field during magnetic resonance imaging at the radiation therapy machine. The MRI scanner may further include a head coil. The magnet of the MRI scanner may include a cryogen-free magnet.

The radiation therapy machine includes an external beam generator and a collimator to generate a radiation beam targeting at portion of the human subject. The radiation therapy machine may be configured to provide external beam therapy.

The platform may include a patient table to which the portion of the human subject is rigidly fixed during transportation from the first position in the magnet to the second position in the radiation therapy machine. The platform may be rotatable over an angular range and may be movable in more than one spatial direction.

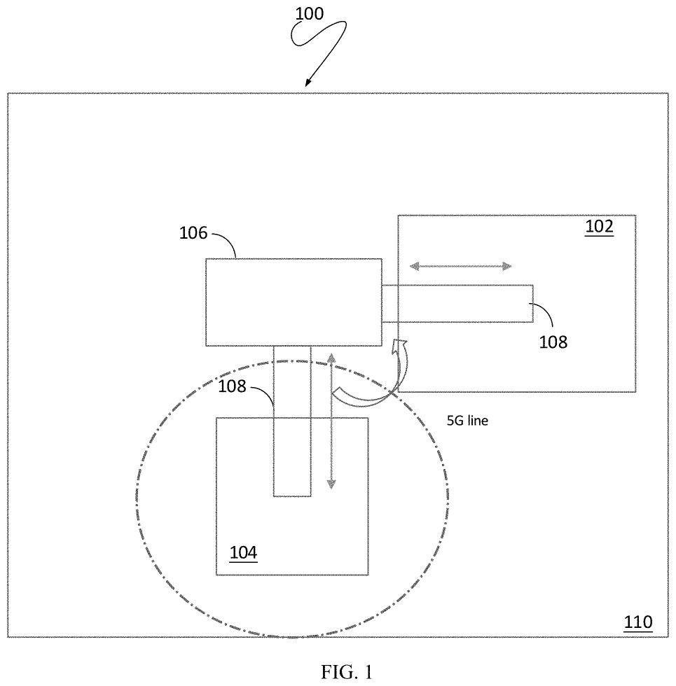

Referring to FIG. 1, this diagram illustrates, in a top view, a combined radiation therapy and MRI system 100 comprising a radiation therapy machine 102 and a MRI scanner 104 adjacent to each other for MRI guided radiation therapy, in accordance with an embodiment of the present disclosure. The system 100 is housed in a radiation therapy bunker 110. Radiation therapy machine 102 comprises an external beam generator, such as a linear accelerator (LINAC), and a multileaf collimator (MLC). The external beam generator generates a radiation beam, such as an X-ray beam, targeted at a particular region of a human subject.

Still referring to FIG. 1, the MRI scanner 104 comprises a magnet, such as a solenoid magnet with an inner bore, to house a human subject for imaging. The magnet has a magnetic field strength in a range of approximately 0.1 Tesla to approximately 7 Tesla for magnetic resonance imaging. The magnet comprises a liquid cryogen-free superconducting magnet. For example, the MRI magnet is cooled with a cold-head helium compressor that is used to cool a supporting structure around which the superconducting wires are wrapped, whereby the magnet is cooled to a temperature below the critical point of a superconducting wire without the use of liquid cryogens, such as liquid helium. A liquid cryogen-free magnet enables operators to manipulate the magnetic field without the need to replenish the cryogen liquid or the risk of quenching the magnet. As a result, an operator can readily reduce or deactivate the magnetic field generated by the magnet.

Still referring to FIG. 1, the MRI scanner 104 further comprises a local coil to image a region of interest of a human subject. In some instances, the local coil comprises a head coil for imaging the head region of a human subject. For example, the close proximity of a local coil provides superior signal-to-noise performance to improve sensitivity or to increase frame rate of a magnetic resonance imaging procedure.

Still referring to FIG. 1, the radiation therapy machine 102 and the MRI scanner 104 are located within the same room. For example, the radiation therapy machine 102 and MRI scanner 104 are located within a radiation therapy bunker, such as a room that having radiation shielding and an appropriate geometric configuration to limit the dose of radiation otherwise experienced outside of the room. The radiation therapy machine 102 and MRI scanner 104 are positioned at an angle relative to each other. The angle comprises a right angle or other configuration. In some instances, the radiation therapy machine 102 and the MRI scanner 104 are disposed facing each other.

Still referring to FIG. 1, the radiation therapy machine 102 is disposed such that the radiation therapy machine 102 is outside of the 5-Gauss line of the magnet when the MRI scanner is operated for imaging, thereby ensuring that the radiation therapy machine 102 is not magnetically attracted by the MRI scanner 104 while the MRI scanner 104 is operated for imaging. In some instances, the radiation therapy machine 102 is disposed even closer to MRI scanner 104. When the MRI scanner is not being used for imaging, and when the radiation therapy machine 102 is being operated for treatment, the magnet of the MRI scanner 104 decreased or deactivated such that the magnetic field generated by the magnet does not affect the operation of the radiation therapy machine 102. For example, the radiation therapy machine 102 and the MRI scanner 104 are disposed relative to each other such that the radiation therapy machine 102 is located outside of the 1-Gauss line when the magnet of the MRI scanner 104 is decreased or deactivated, whereby an undesirable effect of the magnetic field on the radiation therapy machine 102 is prevented while the radiation therapy machine is being used for treatment. In general, the radiation therapy machine 102 and MRI scanner 104 are disposed such that the magnetic field intensity at the radiation therapy machine 102 is below a threshold level when the magnet of the MRI scanner 104 is decreased or deactivated, such as the radiation therapy machine's manufacturer's recommended maximum level of the ambient magnetic field during treatment.

Still referring to FIG. 1, the combined radiation therapy and MRI system 100 further comprises a platform, such as a patient table 108, and a table base 106 on which the patient table 108 is positioned. The patient table 108 is rotatable in relation to the table base 106. The patient table 108 is slidable into the MRI scanner 104 or into the radiation therapy machine 102. In some implementations, the platform may be rotatable over an angular range and is movable in more than more spatial directions. In one example configuration, the platform is rotatable 360°. In another example configuration, the platform is movable in three orthogonal spatial directions. In some implementations, the platform comprises a computerized motor that can be programmed by a computer to perform the rotations and translations.

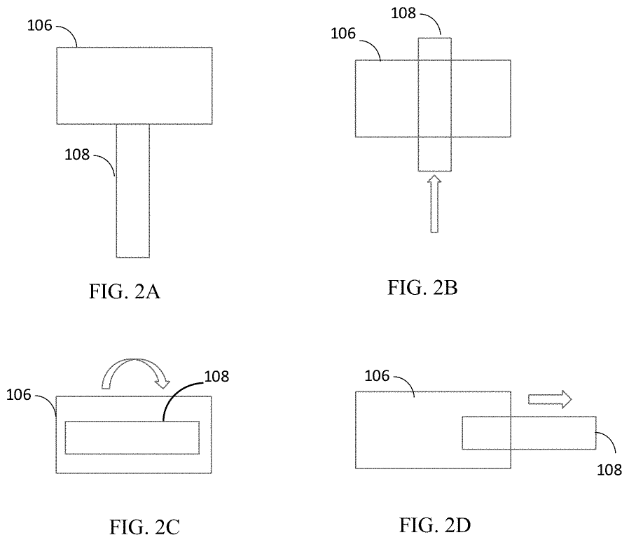

Referring to FIGS. 2A-2D, together, these diagrams illustrate various positions of the patient table 108 operable in a combined radiation therapy and MRI system 100, in accordance with embodiments of the present disclosure. Initially, the patient table 108 is disposed in a first position on the table base 106. Referring to FIG. 2A, this diagram illustrates a first step in the operation of patient table 108, in accordance with an embodiment of the present disclosure. This first position refers to the position for which the patient table 108 is to be inserted, for example, the inner bore of the solenoid magnet of the MRI scanner 104. In this position, a human subject, disposed on the patient table 108, receives an MRI scan, for example, of the head region. MRI images from this position are coregistered with respect to the patient table 108. Referring to FIG. 2B, this diagram illustrates a second step in the operation of patient table 108, in accordance with an embodiment of the present disclosure. When the magnetic resonance imaging is completed, the patient table 108 is moved from the magnet and perpendicularly disposed on the table base 106, e.g., forming a cross configuration with the table base 106. For illustration purposes, only the cross configuration is shown. In other configurations, an oblique angle may be formed. Referring to FIG. 2C, this diagram illustrates a third step in the operation of patient table 108, in accordance with an embodiment of the present disclosure. The patient table 108 is subsequently rotated approximately 90° in relation to the table base 106 such that the patient table 108 aligns with the table base 106. During rotation, the human subject may be immobilized relative to patient table 108 such that the region of interest of the human subject remains fixed relative to the patient table 106. The fixation preserves the coregistration of the region of interest on both the MRI scanner 104 and the radiation therapy machine 102. Referring to FIG. 2D, this diagram illustrates a fourth step in the operation of patient table 108, in accordance with an embodiment of the present disclosure. After rotation, the patient table 108 is moved in relation to the table base 106 and into the radiation therapy machine 102 for the human subject to receive radiation therapy.

Still referring to FIGS. 2A-2D, together, during use of the combined radiation therapy and MRI system 100, a human subject is disposed on the patient table 108. The patient table 108 is moved into a first position such that at least a portion of the human subject is located inside the magnet of the MRI scanner 104 and, therefore, is imaged by the MRI scanner 104. The MRI scanner 104 is operated while the patient table 108 is in the first position to obtain an image of the human subject. The patient table 108 is moved into a second position such that the human subject is positioned to receive radiation therapy from the radiation therapy machine 102; and the magnetic field generated by the magnet of the MRI scanner 104 is reduced such that the magnetic field at the radiation therapy machine 102 is below a threshold value (for example, a value at which the magnetic field does not affect the operation of the linear accelerator or other equipment of the radiation therapy machine 102). While the patient table 108 is in the second position and the magnetic field at the radiation therapy machine is below the threshold value, the radiation therapy machine is operated to perform radiation therapy on the human subject.

Still referring to FIGS. 2A-2D, together, in the implementations incorporating a computerized motor, the motor is programmed by a computer to cause patient table 108 to perform rotations and translations so that the human subject can be expediently moved from the MRI scanner 104 to the radiation therapy machine 102. In these implementations, the computer may execute a program to automatically move the patient table 108 away from the MRI scanner 104 (for example, out of the inner bore of the main magnet), rotate the patient table 108 to align the patient table 108 with the radiation therapy machine 102, and then move the patient table 108 into the radiation therapy machine 102 for the human subject to receive radiation therapy.

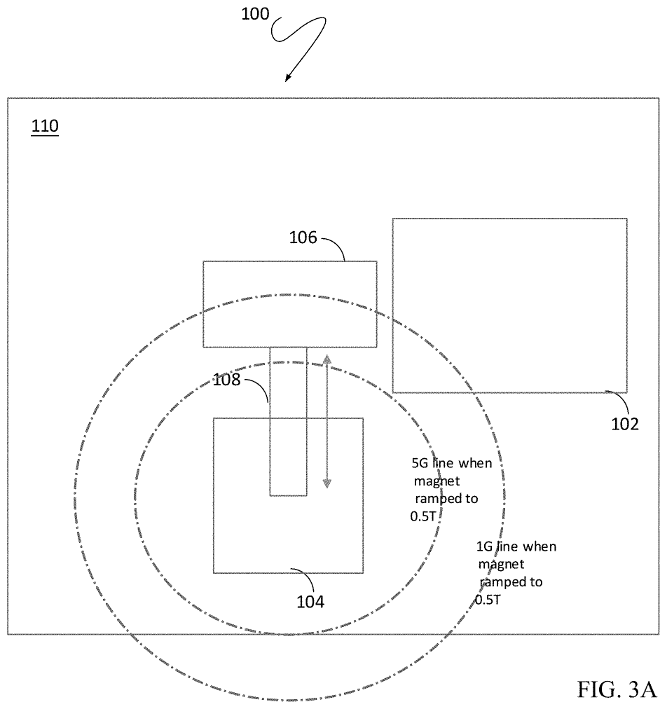

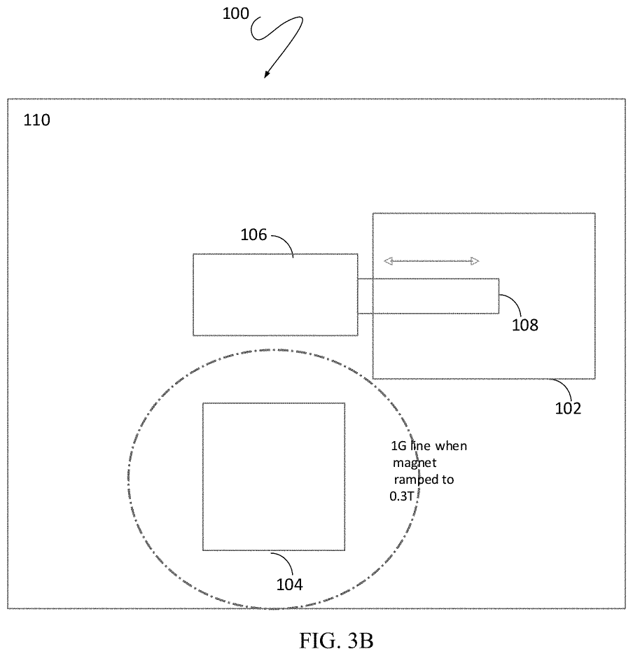

Referring to FIGS. 3A and 3B, together, these diagrams illustrate a change in magnetic field intensity of the magnetic field produced by the MRI scanner 104 of a combined radiation therapy and MRI system 100 when switching from an imaging mode, using the MRI scanner 104, to treatment mode, using the radiation therapy machine 102, in accordance with embodiments of the present disclosure. Referring to FIG. 3A, the patient table 108 is configured to accommodate a patient (not shown) and is disposed in a first position in which at least a portion of the patient is located in the MRI scanner 104 for imaging. In this example, the magnet of the MRI scanner 104 is set to 0.5 T when operating the MRI scanner 104 for imaging. The radiation therapy machine 102 and the MRI scanner 104 are arranged relative to each other such that the radiation therapy machine 102 is disposed outside of the 5-Gauss line when the magnet is set to 0.5 T, but a portion of the radiation therapy machine 102 is disposed within the 1-Gauss line.

Still referring to FIGS. 3A and 3B, together, if the radiation therapy machine 102 is, otherwise, operated while within the 1-Gauss line, for example, the magnetic field interacts with and disturbs the operation of the accelerator on the radiation therapy machine 102. Specifically, the accelerator comprises a linear accelerator (LINAC) operable to emit high-energy x-rays to destroy tumors. In more detail, these high-energy x-rays are generated by using microwave technology to accelerate electrons in a part of the LINAC device called the wave guide. These accelerated electrons are then aimed at a heavy metal target, such as tungsten, to cause plenty of collisions. High-energy x-rays are produced from the target as a result of these collisions. If the LINAC is activated while within the 1-Gauss (or greater) field, the magnetic field interferes with the LINAC to the detriment of the operation of the LINAC. Embodiments of the present disclosure are configured to reduce or eliminate such interference.

Still referring to FIGS. 3A and 3B, together, to reduce or eliminate such interference, the magnetic field intensity of the magnet of the MRI scanner 104 is decreased when the radiation therapy machine 102 is used in the same radiation therapy bunker 110. In the example shown, the patient table 108 is moved from the first position to a second position in which the patient can receive therapy from the radiation therapy machine 102. Before such therapy is initialized, the magnetic field of the magnet of the MRI scanner 108 is decreased, for example, to approximately 0.3 T, thereby resulting in the radiation therapy machine 102 being effectively disposed outside the 1-Gauss line. When the LINAC of radiation therapy machine 102 is operated at this point, the magnetic field intensity may be sufficiently reduced at the radiation therapy machine 102 such that the magnetic field does not interfere with the operation of the LINAC. In some implementations, the magnet of the MRI scanner 108 is deactivated.

Still referring to FIGS. 3A and 3B, together, the sequence for decreasing or deactivating the magnetic field intensity of the magnet is initiated while the patient table 108 is being moved from the magnet. In other words, decreasing or deactivating the magnetic field intensity of the magnet coordinated with transitioning the patient table 108 from the first position to the second position. In one instance, reducing the magnetic field intensity from approximately 0.5 T to approximately 0.3 T has a duration of approximately 1 minute to approximately 30 minutes, thereby overlapping with the time period for moving the patient table 108 from the MR scanner 104 and then into the radiation therapy machine 102. In this instance, an operator may reduce the magnetic field intensity of the cryogen-free magnet without having to deactivate the magnet or replenish liquid helium.

Still referring to FIGS. 3A and 3B, together, in the implementations incorporating a motor that is controlled by a computer program, the program may also initiate a sequence to decrease or deactivate the magnetic field intensity of the main magnet of the MRI scanner 104. The program may further automatically activate the external beam generator (for example, a LINAC) of the radiation therapy machine 102 when the interference, caused by the main magnet, has been substantially suppressed. In some implementations, a magnetic field measuring device, such as a Gauss meter, may be disposed at the perimeter of the radiation therapy machine 102 to detect and measure the fringe field. The measuring device may be coupled to the computer, e.g., the same computer controlling the motorized platform, as shown in FIG. 1. The measuring device provides digitized input to the computer controlling the sequence of operating the radiation therapy machine 102. For example, when the fringe field at the radiation therapy machine 102 has decreased within a threshold value, the sequence of operating the radiation therapy machine 102 may initiate. Accordingly, the process of transferring the human subject from MRI scanner 104 to the radiation therapy machine 102 can be automated.

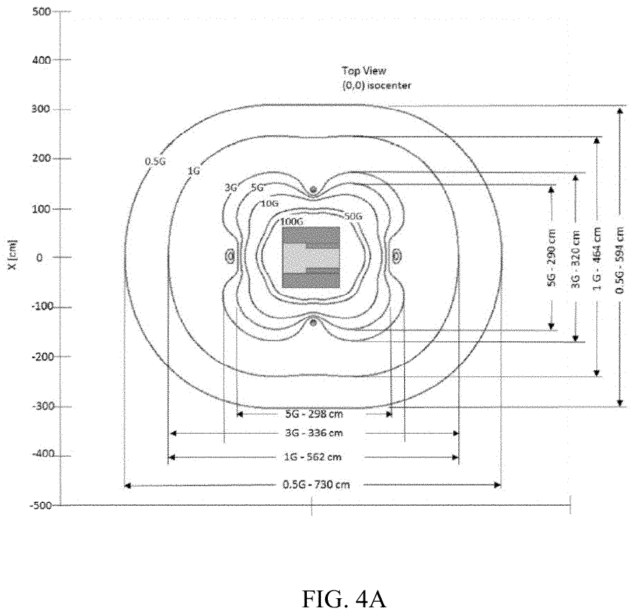

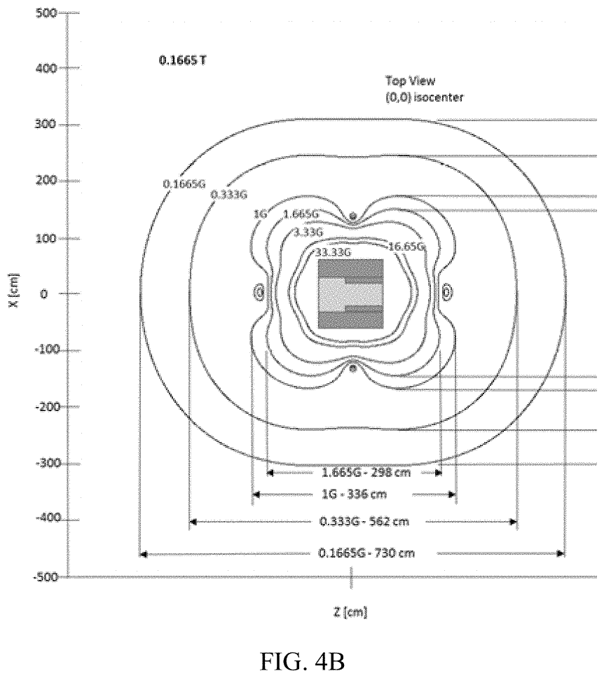

Referring to FIG. 4A, this diagram illustrates a fringe field for a 0.5-Tesla magnet, in accordance with an embodiment of the present disclosure. The 5-Gauss line is approximately 1.45 m away from the magnet center while the 1-Gauss line is approximately 2.32 m away from the magnet. Referring to FIG. 4B, for comparison, the fringe field is illustrated for a 0.1665-Tesla magnet in which the 1-Gauss line is only 1.68 m away from the magnet center. Because the reduced magnetic field has fringe field that is decreased, operating an accelerator for radiation therapy at the same physical distance from the magnet is enabled as such operation is less hindered and more advantageous. In some implementations, the separation distance between the MR scanner 104 and the radiation therapy machine 102 may be no less than 1.68 m. In some implementations, the fringe field at the radiation therapy machine 102 comprises a range of less than approximately 1 Gauss when the LINAC of the radiation therapy machine 102 is operated. In some implementations, the fringe field at the radiation therapy machine 102 comprises a range of less than approximately 60% of the field strength during MRI operation when the magnet is operated at its full strength.

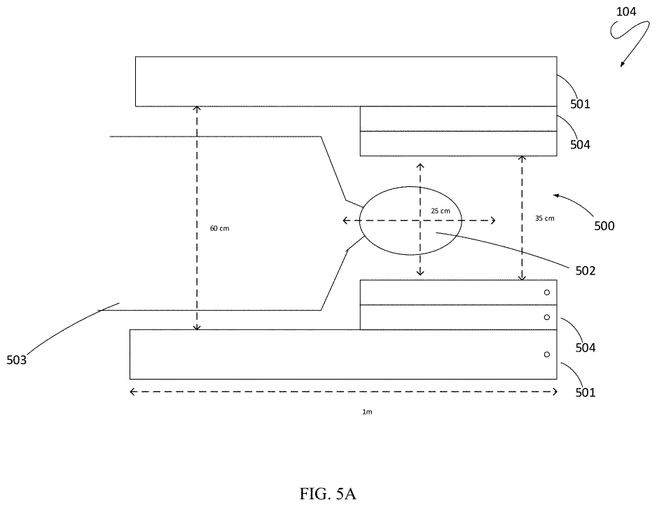

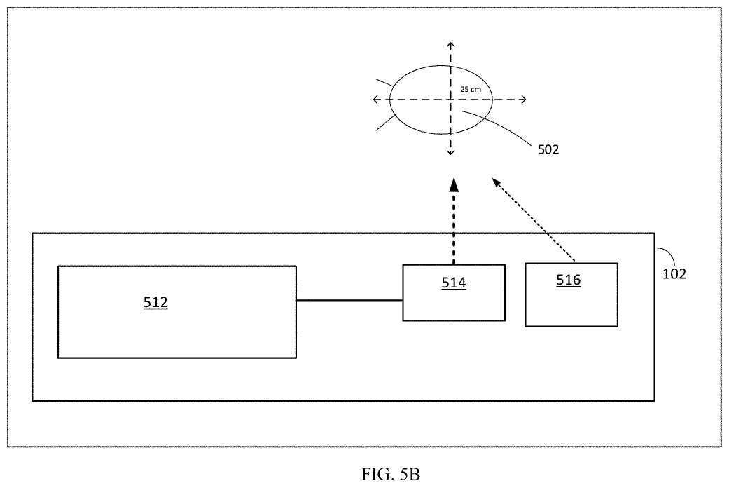

Referring to FIG. 5A, this diagram illustrates a configuration for imaging the head region 502 of a human subject 503 in the MRI scanner 104, in accordance with an embodiment of the present disclosure. This example shows a cross-sectional view of the MRI scanner 104 with the sidewalls of a solenoid magnet 501 enclosing the inner bore 500. The example dimensions are shown in relation to the relative sizes of the head region and inner bore 500 of solenoid magnet 501. The inner bore 500 of solenoid magnet 501 comprises a width of approximately 60 cm. The diameter of the inner bore 500 comprises a size which can be larger than the width, for example, approximately 70 cm wide. The inner bore 500 houses a coil assembly 504 comprising a gradient coil. The gradient coil provides a perturbation of the magnetic field to encode MRI signals emitted from the head region 502 of the human subject 503.

Still referring to FIG. 5A, in some implementations, the coil assembly 504 comprises a radio-frequency (RF) transmit coil that transmits RF excitation pulses into the head region 502 of the human subject 503. The inner diameter of coil assembly 504 may be reduced to a value that accommodates the insertion of the head region 502, but is, for example, less than approximately 60 cm, less than approximately 50 cm, less than approximately 45 cm, less than approximately 40 cm, and less than approximately 35 cm. The coil assembly 504 is recessed within the solenoid magnet 501, such that the patient body, e.g., the shoulders, may be inserted within a broader diameter region, for example, having a diameter of approximately 60 cm, associated with the coil assembly 504, while head region 502 may be inserted within a narrower diameter region associated with the coil assembly 504. In these implementations, the resulting MRI signals are received by a head coil (not shown) surrounding the head region 502. In other implementations, the head coil operates as both transmit-coil and receive-coil. The close proximity of the head coil to the head region 502 provides superior signal-to-noise performance.

Referring to FIG. 5B, this diagram illustrates a configuration for targeting an external beam therapy, such as a conventional X-ray radiation beam, to the same head region 502 of the human subject 503, in accordance with an embodiment of the present disclosure. Because the head region 502 of the human subject 503 is secured relative to the patient table 108, the coregistration is maintained from the first position in the MRI scanner 104 to the second position in the radiation therapy machine 102 such that targeting the x-ray beam can be based on the magnetic resonance images recently obtained from the same region.

Still referring to FIG. 5B, the radiation therapy machine 102 comprises a LINAC 512, a collimator 514, and an image intensifier 516. The LINAC 512 includes, for example, a microwave waveguide to accelerate electrons aimed at, for example, a tungsten target. As the high energy electrons collide with the tungsten target, high-energy x-rays are produced. To direct the resulting X-ray radiation beam to the head region 502, the collimator 514, such as a multi-leaf collimator (MLC), collimates the X-ray beam onto a specific target area as guided by, for example, the magnetic resonance images of the head region 502, as obtained from the MRI scanner 104 when the head region 502 of the human subject 503 was therein disposed. In some instances, targeting the x-ray beam at the specific region for radiation therapy is programmed in accordance with guidance information provided by the magnetic resonance images. Some implementations involve using an external beam generator, other than LINAC 512. For context, an external beam generator refers to a radiation therapy apparatus that provides a therapeutic radiation energy source. Example external beam generators include a LINAC, a proton beam generator, or a gamma knife generator. Example gamma knife generators include cobalt-60 sources, cesium-137 sources, europium-152 sources, or radium-226 sources.

Still referring to FIG. 5B, the image intensifier 516 comprises a fluoroscopy device to generate real-time x-ray images of the head region 502 to provide intraoperative guidance information for placing the targeting x-ray beams. In some implementations, the fluoroscopic images of the head region 502 provide the general layout, including the boundaries of the skull. The images obtained from MRI scanner 104 immediately before the radiation therapy may be fused with the CT images or the x-ray images. Fusing the MRI image on the fluoroscopic images comprises aligning the MRI images with the fluoroscopic images by virtue of fixing the region of interest of the head region 502 with respect to patient table 108. Fusing the MRI images on the fluoroscopic images comprises using fiducial markers that are visible in both MRI and fluoroscopy. Fusing more than one set of images comprises displaying one set of images superimposed on another set of images, for example, with a transparency configuration.

Still referring to FIG. 5B, the images obtained from MRI scanner 104 may provide superior soft-tissue contrast, delineating fine details of the lobular and ventricular structures of the brain encompassing both gray matter and white matter. The images obtained from MRI scanner 104 may include a contrast type tailored to highlight, for example, the boundary of a tumor in the brain. The contrast may include, for example, T1-weighting, T2-weighting, diffusion-weighted, magnetization-transfer weighted, etc. The images obtained from MRI scanner 104 may include a 3-dimensional image of head region 502. The images obtained from MRI scanner 104 comprise a multi-slice image of head region 502. Upon completion of radiation therapy, LINAC 512 of radiation therapy machine 102 is deactivated; and the magnetic field intensity of the magnet of the MRI scanner 104 is then increased. In some instances, increasing the magnetic field intensity from a reduced magnetic field is more expeditious than increasing the magnetic field intensity from a deactivated state.

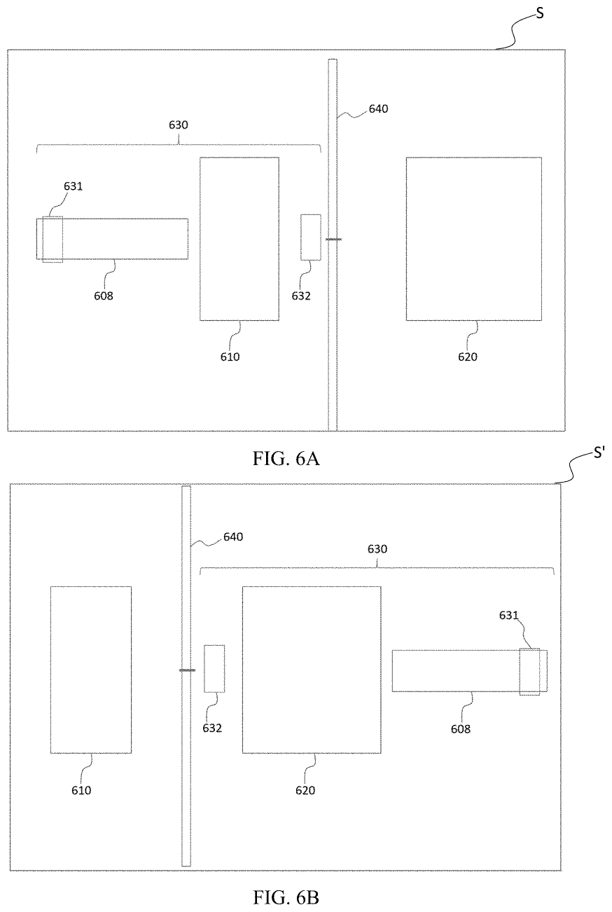

Referring to FIG. 6A, this schematic diagram illustrates, in a top view, an MRI-CT system S for sequentially imaging, in accordance with an embodiment of the present disclosure. The MRI-CT system S comprises: a CT component 610, such as a CT scanner; an MR component 620, such as an MR scanner; a transporter 608; a bridge 630 configured to engage the transporter 608 and disposable between the CT component 610 and the MR component 620; and a movable barrier 640, such as a removable barrier or door, disposable between the CT component 610 and the MR component 620. The movable barrier 640 comprises a magnetic shield. The CT component 610 and the MR component 620 are disposable in relation to one another in at least one of linearly aligned and colinearly aligned. The CT component 610, comprising the CT scanner, has a CT scanner limit of approximately 1 Gauss; however, for at least that the CT component 610 is operable with the movable barrier 640 comprising the magnetic shield and that the MR component 620 comprises an MR scanner having a small fringe field in a range of approximately 60 cm to approximately 300 cm for the distance of the 5-Gauss level as measured from a center of the MR component 620 in the absence of a magnetic shield, the MR component 620 is operable despite the CT scanner limit, thereby overcoming many challenges in the related art. The magnetic shield comprises at least one shield layer. The at least one shield layer comprises at least one magnetic shielding layer. The at least one magnetic shielding layer comprises a magnetic shielding material. The magnetic shielding material comprises a ferromagnetic material. The ferromagnetic material comprises at least one of silicon steel and any other iron alloy.

Still referring to FIG. 6A, in an embodiment of the MRI-CT system S, the movable barrier 640 comprises a RF shielding feature, such as an RF shield, the movable barrier 640 operating, at least in part, to protect the MR component 620 against RF noise emanating from the CT component 610 via the RF shielding feature. The bridge 630 is configured to engage the transporter 608 and to facilitate coupling the transporter 608 with the MR component 620 by way of at least one MR component coupler (not shown), such as a latching mechanism, e.g., disposed proximate, or within, the MR component 620. The movable barrier 640 further comprises at least one of at least one window (not shown) and at least one waveguide (not shown). The RF shielding feature protects the MR component 620 against RF noise emanating from the CT component 610 during MRI imaging. For most implementations of the system S, the movable barrier 640 is disposed in an open position during MRI scanning by the MR component 620 and in a closed position during CT scanning by the CT component 610; however, for other implementations of the system S, the movable barrier 640 is disposed either an open position or a closed position during MRI scanning by the MR component 620 and in a closed position during CT scanning by the CT component 610. In some embodiments of the system S, the bridge 630 comprises a plurality of portions, such as a first portion 631 and a second portion 632, wherein each portion comprises at least one waveguide configured to at least one of couple with at least one cable of the transporter 608 and accommodate least one cable of the transporter 608. The at least one shield layer further comprises at least one RF shield layer. The at least one RF shield layer comprises an RF shielding material. The RF shielding material comprises copper.

Referring to FIG. 6B, this schematic diagram illustrates, in a top view, an MRI-CT system S′ for sequentially imaging, in accordance with an alternative embodiment of the present disclosure. In this alternative embodiment, the transporter 608 is initially disposable in an alternative “home” position, wherein the alternative home position is located proximate the MR component 620 and on an opposite side (in relation to the position shown in FIG. 6A) of the MR component 620, and wherein the transporter 608 is operable to move a patient (not shown) from the MR component 620 to the CT component 610. The MRI-CT system S′ comprises: a CT component 610, such as a CT scanner; an MR component 620, such as an MR scanner; a transporter 608; a bridge 630 configured to engage the transporter 608 and disposable in relation to the MR component 620; and a movable barrier 640, such as a removable barrier or door, disposable between the CT component 610 and the MR component 620. The movable barrier 640 comprises a magnetic shield. The CT component 610 and the MR component 620 are disposable in relation to one another in at least one of linearly aligned and colinearly aligned. The CT component 610, comprising the CT scanner, has a CT scanner limit of approximately 1 Gauss; however, for at least that the CT component 610 is operable with the movable barrier 640 comprising the magnetic shield and that the MR component 620 comprises an MR scanner having a small fringe field in a range of approximately 60 cm to approximately 300 cm for the distance of the 5-Gauss level as measured from a center of the MR component 620 in the absence of a magnetic shield, the MR component 620 is operable despite the CT scanner limit, thereby overcoming many challenges in the related art. The magnetic shield comprises at least one shield layer. The at least one shield layer comprises at least one magnetic shielding layer. The at least one magnetic shielding layer comprises a magnetic shielding material. The magnetic shielding material comprises a ferromagnetic material. The ferromagnetic material comprises at least one of silicon steel and any other iron alloy.

Still referring to FIG. 6B, in an embodiment of the MRI-CT system S′, the movable barrier 640 comprises a RF shielding feature, such as an RF shield, the movable barrier 640 operating, at least in part, to protect the MR component 620 against RF noise emanating from the CT component 610 via the RF shielding feature. The bridge 630 is configured to engage the transporter 608 and to facilitate coupling the transporter 608 with the MR component 620 by way of at least one MR component coupler (not shown), such as a latching mechanism, e.g., disposed proximate, or within, the MR component 620. The movable barrier 640 further comprises at least one of at least one window (not shown) and at least one waveguide (not shown). The RF shielding feature protects the MR component 620 against RF noise emanating from the CT component 610 during MRI imaging. For most implementations of the system S′, the movable barrier 640 is disposed in an open position during MRI scanning by the MR component 620 and in a closed position during CT scanning by the CT component 610; however, for other implementations of the system S′, the movable barrier 640 is disposed either an open position or a closed position during MRI scanning by the MR component 620 and in a closed position during CT scanning by the CT component 610. In some embodiments of the system S′, the bridge 630 comprises a plurality of portions, such as a first portion 631 and a second portion 632, wherein each portion comprises at least one waveguide configured to at least one of couple with at least one cable of the transporter 608 and accommodate least one cable of the transporter 608. The at least one shield layer further comprises at least one RF shield layer. The at least one RF shield layer comprises an RF shielding material. The RF shielding material comprises copper.

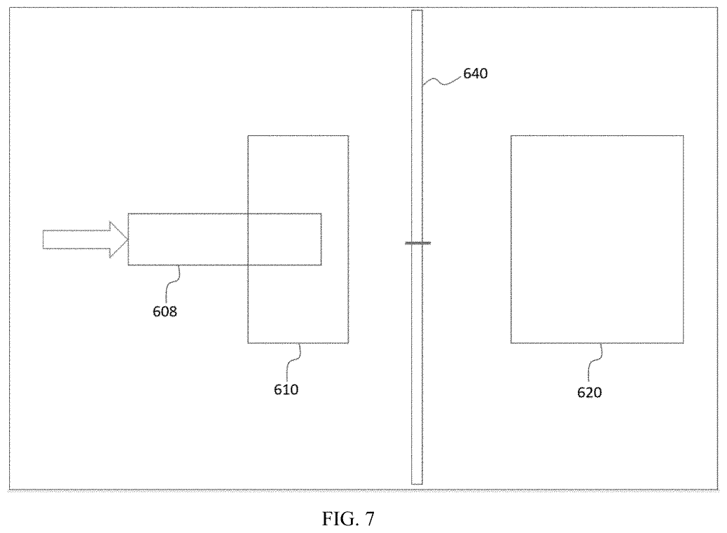

Referring to FIG. 7, this schematic diagram illustrates, in a top view, the MRI-CT system S for sequentially imaging, operating in a CT imaging mode, in accordance with an embodiment of the present disclosure. A patient (not shown) is disposed on the transporter 608, wherein the transporter 608 is disposed on the first portion 631 of the bridge 630. The first portion 631 of the bridge 630 is configured to firstly move the transporter 608 to, and through, the CT component 610.

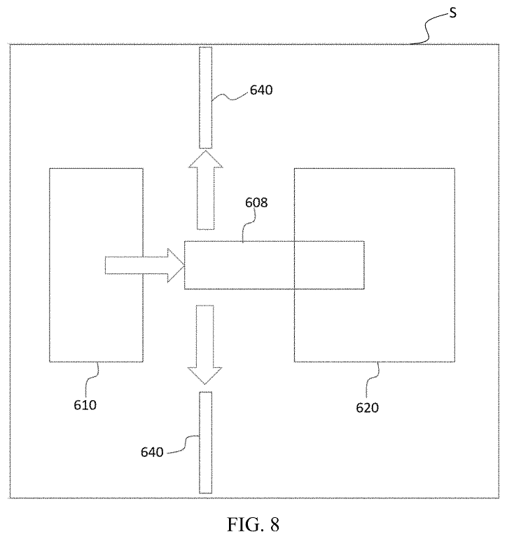

Referring to FIG. 8, this schematic diagram illustrates, in a top view, the MRI-CT system S for sequentially imaging, operating in an MR imaging mode, in accordance with an embodiment of the present disclosure. The patient (not shown) is disposed on the transporter 608, wherein the transporter 608 is disposed on the second portion 632 of the bridge 630. The second portion 632 of the bridge 630 is configured to secondly move the transporter 608 to, and, at least partially, into, the MR component 620.

Still referring to FIG. 8 and referring back to FIG. 7, the MRI-CT system S for sequentially imaging operates in a sequence or in series, e.g., by firstly or initially CT scanning the patient via the CT component 610 and secondly or subsequently MR scanning the patient via the MR component 620. At least one of the bridge 630 and the transporter 608 comprises a moving feature, such as a conveyor belt and a movable platform, to facilitate moving the transporter 608 forward in a sequence and backward from the sequence. The transporter 608 comprises a material that is compatible with both CT scanning requirements of the CT component 610 as well as MR scanning requirements of the MR component 620, whereby the related art challenge of materials incompatibility is overcome. For instance, the transporter 608 comprises, at least partially, fiberglass, e.g., for a section of the transporter 608 on which the patient's head and shoulders would be disposed; and only such fiberglass section would be disposed in the MR component 620 during operation of the system S in the MR imaging mode. Even if a remaining section of the transporter 608 on which the patient's torso, arms, and legs would be disposed comprises carbon fiber, the section of the transporter 608, comprising fiberglass, would mitigate or eliminate many challenges in the related art.

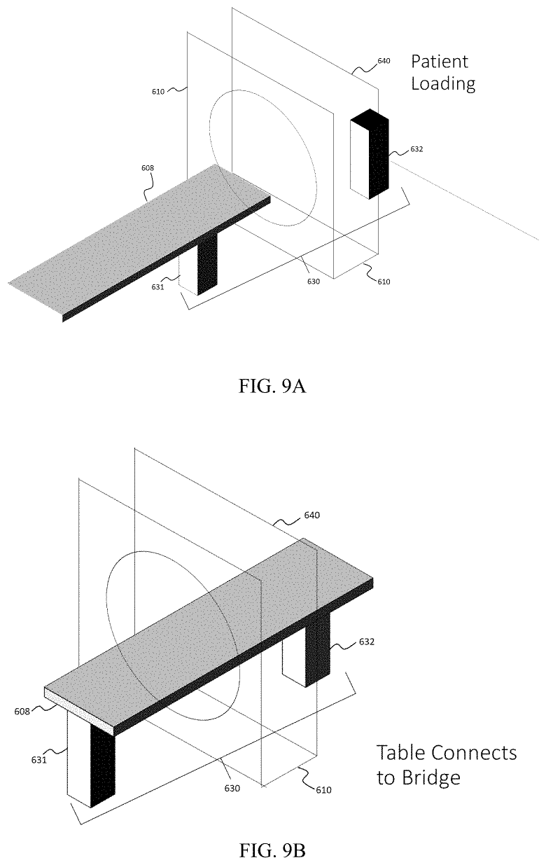

Referring to FIGS. 9A and 9B, these schematic diagrams illustrate, in a perspective view, the transporter 608, engaged with the first portion 631 of the bridge 630 and the second portion 632 of the bridge 630, for moving the patient (not shown) through the CT component 610 of an MRI-CT system S for sequentially imaging, as shown in FIGS. 6 and 7, in accordance with an embodiment of the present disclosure.

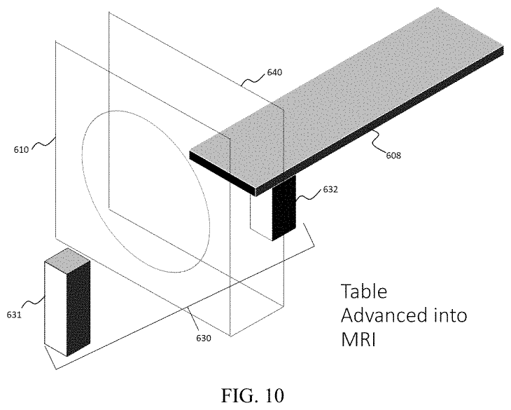

Referring to FIG. 10, this schematic diagram illustrates, in a perspective view, the transporter 608, engaged with the second portion 632 of the bridge 630, for moving the patient (not shown) to and into the MR component 620 of an MRI-CT system S for sequentially imaging, as shown in FIGS. 7 and 8, in accordance with an embodiment of the present disclosure.

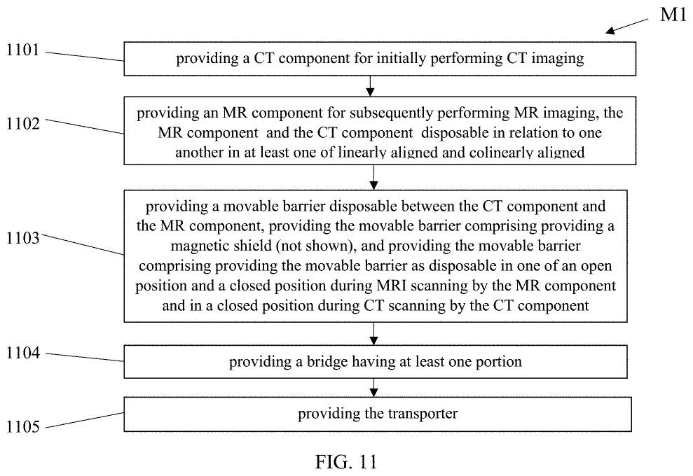

Referring to FIG. 11, this flow diagram illustrates a method M1 of providing an MRI-CT system S for sequentially imaging a subject (not shown), in accordance with an embodiment of the present disclosure. The method M1 comprises: providing a CT component 610 for initially performing CT imaging, as indicated by block 1101; providing an MR component 620 for subsequently performing MR imaging, the MR component 620 and the CT component 610 disposable in relation to one another in at least one of linearly aligned and colinearly aligned, as indicated by block 1102; and providing a movable barrier 640 disposable between the CT component 610 and the MR component 620, providing the movable barrier 640 comprising providing a magnetic shield (not shown), and providing the movable barrier 640 comprising providing the movable barrier 640 as disposable in one of an open position and a closed position during MRI scanning by the MR component 620 and in a closed position during CT scanning by the CT component 610, as indicated by block 1103.

Still referring to FIG. 11, the method M1 further comprises providing a bridge 630 having at least one portion, as indicated by block 1104. Providing the bridge 630, as indicated by block 1104, comprises configuring the bridge 630 to engage a transporter 608; and providing the bridge 630, as indicated by block 1104, comprises configuring the bridge 630 as disposable between the CT component 610 and the MR component 620, as indicated by block 1104. The method M1 further comprises providing the transporter 608, as indicated by block 1105. Providing the transporter 608, as indicated by block 1105, comprises configuring the transporter 608 to engage the bridge 630, wherein at least one of providing the bridge, as indicated by block 1104, and providing the transporter, as indicated by block 1105, comprises providing a moving feature to facilitate moving the transporter forward in a sequence and backward from the sequence.

Still referring to FIG. 11, in the method M1, providing the CT component 610, as indicated by block 1101, comprises providing a CT scanner having a CT scanner limit of approximately 1 Gauss; and providing the MR component 620, as indicated by block 1102, comprises providing an MR scanner having a small fringe field in a range of approximately 60 cm to approximately 300 cm for the distance of the 5-Gauss level as measured from a center of the MR component 620 in the absence of a magnetic shield, whereby the MR component 620 is operable regardless of the CT scanner limit of the CT scanner.

Still referring to FIG. 11, in the method M1, providing the movable barrier 640, as indicated by block 1103, comprises providing at least one of at least one removable barrier, at least one door, at least one window, and at least one waveguide; and providing the movable barrier further comprises providing a radio-frequency (RF) shield, whereby the movable barrier 640, via the RF shield, operating, at least in part, protects the MR component 620 against RF noise emanating from the CT component 610 during MRI imaging.

Still referring to FIG. 11, in the method M1, providing the bridge 630, as indicated by block 1104, further comprises configuring the bridge 630 to facilitate coupling the transporter 608 with the MR component 620 by way of at least one MR component coupler (not shown). Providing the bridge having the at least one portion, as indicated by block 1104, comprises providing a plurality of portions; and providing the plurality of portions comprises providing each portion of the plurality of portions with at least one waveguide (not shown) configured to at least one of: couple with at least one cable (not shown) of the transporter 608; and accommodate at least one cable (not shown) of the transporter 608.

Still referring to FIG. 11, in the method M1, providing the bridge 630 having the at least one portion, as indicated by block 1104, comprises providing a plurality of portions, wherein providing the plurality of portions comprises providing a first portion 631 and providing a second portion 632, wherein providing the first portion 631 comprises configuring the first portion 631 to firstly move the transporter 608 to, and through, the CT component 610 when the transporter 608 is disposed in relation to the first portion 631, and wherein providing the second portion 632 comprises configuring the second portion 632 to secondly move the transporter 608 to, and, at least partially, into, the MR component 620 when the transporter 608 is disposed is disposed in relation to the second portion 632.

Still referring to FIG. 11, in the method M1, providing the transporter, as indicated by block 1105, comprises providing a material being compatible with CT scanning requirements of the CT component and MR scanning requirements of the MR component 620, wherein providing the transporter, as indicated by block 1105, comprises providing a first section 608a and providing a second section 608b, wherein providing the first section 608a comprises providing the first section 608a corresponding to a location for accommodating a head and shoulders of the subject (not shown), wherein providing the second section 608b comprises providing the second section 608b corresponding to a location for accommodating a torso of the subject (not shown), wherein providing the first section 608a comprises providing fiberglass, and wherein providing the first section 608a comprises configuring the first section 608a for disposition in the MR component 620 during operation of the system S in an MR imaging mode.

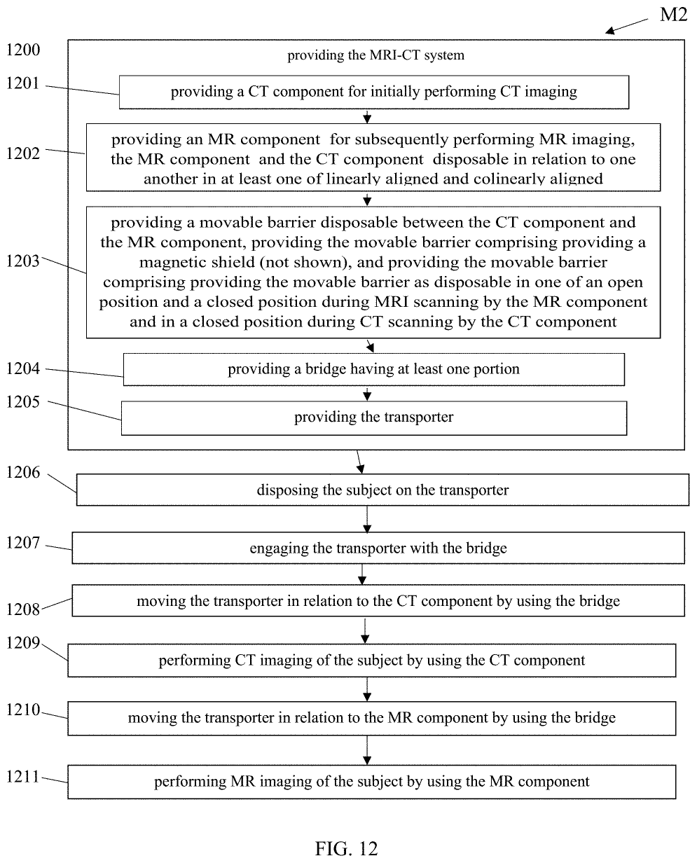

Referring to FIG. 12, this flow diagram illustrates a method M2 of sequentially imaging by way of an MRI-CT system S, in accordance with an embodiment of the present disclosure. The method M2 comprises: providing the MRI-CT system S, as indicated by block 1200, comprising: providing a CT component 610 for initially performing CT imaging, as indicated by block 1201; providing an MR component 620 for subsequently performing MR imaging, the MR component 620 and the CT component 610 disposable in relation to one another in at least one of linearly aligned and colinearly aligned, as indicated by block 1202; and providing a movable barrier 640 disposable between the CT component 610 and the MR component 620, providing the movable barrier 640 comprising providing a magnetic shield (not shown), and providing the movable barrier 640 comprising providing the movable barrier 640 as disposable in one of an open position and a closed position during MRI scanning by the MR component 620 and in a closed position during CT scanning by the CT component 610, as indicated by block 1203; providing a bridge 630 having at least one portion, providing the bridge comprising configuring the bridge 630 to engage a transporter 608, and providing the bridge 630 comprising configuring the bridge 630 as disposable between the CT component 610 and the MR component 620, as indicated by block 1204; providing the transporter 608, providing the transporter 608 comprising configuring the transporter 608 to engage the bridge 630, wherein at least one of providing the bridge, as indicated by block 1204, and providing the transporter, as indicated by block 1205, comprises providing a moving feature (not shown) to facilitate moving the transporter 608 forward in a sequence and backward from the sequence, as indicated by block 1205; disposing the subject (not shown) on the transporter 608, as indicated by block 1206; engaging the transporter 608 with the bridge 630, as indicated by block 1207; moving the transporter 608 in relation to the CT component 610 by using the bridge 630, as indicated by block 1208; performing CT imaging of the subject by using the CT component, as indicated by block 1209; moving the transporter 608 in relation to the MR component 620 by using the bridge 630, as indicated by block 1210; and performing MR imaging of the subject by using the MR component 620, as indicated by block 1211. In some embodiments of the present disclosure, the method M2, providing the MRI-CT system S, as indicated by block 1200, further comprises one of: disposing the CT component and the MR component in the same room; and disposing the CT component and the MR component in adjacent rooms with a magnetically shield door disposed therebetween.

Still referring to FIG. 12, the method M2 further comprises providing a bridge 630 having at least one portion, as indicated by block 1204. Providing the bridge 630, as indicated by block 1204, comprises configuring the bridge 630 to engage a transporter 608; and providing the bridge 630, as indicated by block 1204, comprises configuring the bridge 630 as disposable between the CT component 610 and the MR component 620, as indicated by block 1204. The method M1 further comprises providing the transporter 608, as indicated by block 1205. Providing the transporter 608, as indicated by block 1205, comprises configuring the transporter 608 to engage the bridge 630, wherein at least one of providing the bridge, as indicated by block 1204, and providing the transporter, as indicated by block 1205, comprises providing a moving feature to facilitate moving the transporter forward in a sequence and backward from the sequence.

Still referring to FIG. 12, in the method M2, providing the CT component 610, as indicated by block 1201, comprises providing a CT scanner having a CT scanner limit of approximately 1 Gauss; and providing the MR component 620, as indicated by block 1202, comprises providing an MR scanner having a small fringe field in a range of approximately 60 cm to approximately 300 cm for the distance of the 5-Gauss level as measured from a center of the MR component 620 in the absence of a magnetic shield, whereby the MR component 620 is operable regardless of the CT scanner limit of the CT scanner.

Still referring to FIG. 12, in the method M2, providing the movable barrier 640, as indicated by block 1203, comprises providing at least one of at least one removable barrier, at least one door, at least one window, and at least one waveguide; and providing the movable barrier further comprises providing a radio-frequency (RF) shield, whereby the movable barrier 640, via the RF shield, operating, at least in part, protects the MR component 620 against RF noise emanating from the CT component 610 during MRI imaging.

Still referring to FIG. 12, in the method M2, providing the bridge 630, as indicated by block 1204, further comprises configuring the bridge 630 to facilitate coupling the transporter 608 with the MR component 620 by way of at least one MR component coupler (not shown). Providing the bridge having the at least one portion, as indicated by block 1204, comprises providing a plurality of portions; and providing the plurality of portions comprises providing each portion of the plurality of portions with at least one waveguide (not shown) configured to at least one of: couple with at least one cable (not shown) of the transporter 608; and accommodate at least one cable (not shown) of the transporter 608.

Still referring to FIG. 12, in the method M2, providing the bridge 630 having the at least one portion, as indicated by block 1204, comprises providing a plurality of portions, wherein providing the plurality of portions comprises providing a first portion 631 and providing a second portion 632, wherein providing the first portion 631 comprises configuring the first portion 631 to firstly move the transporter 608 to, and through, the CT component 610 when the transporter 608 is disposed in relation to the first portion 631, and wherein providing the second portion 632 comprises configuring the second portion 632 to secondly move the transporter 608 to, and, at least partially, into, the MR component 620 when the transporter 608 is disposed is disposed in relation to the second portion 632.

Still referring to FIG. 12, in the method M2, providing the transporter, as indicated by block 1105, comprises providing a material being compatible with CT scanning requirements of the CT component and MR scanning requirements of the MR component 620, wherein providing the transporter, as indicated by block 1205, comprises providing a first section 608a and providing a second section 608b, wherein providing the first section 608a comprises providing the first section 608a corresponding to a location for accommodating a head and shoulders of the subject (not shown), wherein providing the second section 608b comprises providing the second section 608b corresponding to a location for accommodating a torso of the subject (not shown), wherein providing the first section 608a comprises providing fiberglass, and wherein providing the first section 608a comprises configuring the first section 608a for disposition in the MR component 620 during operation of the system S in an MR imaging mode.

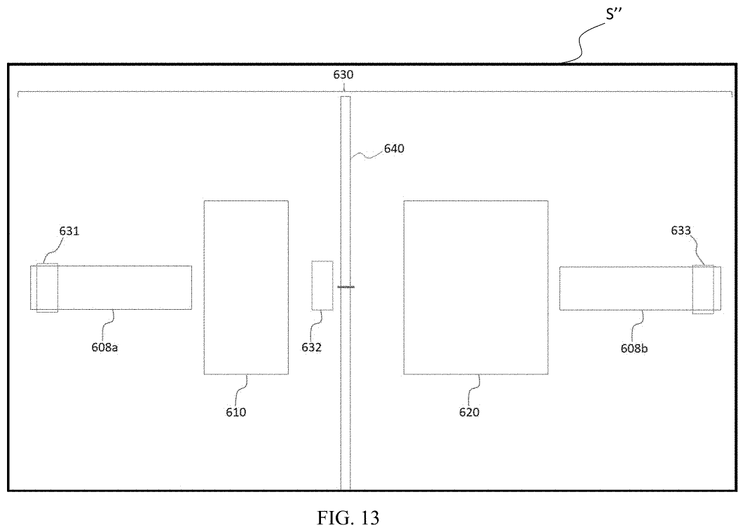

Referring to FIG. 13, this diagram illustrates, in a top view, an CT-PET/CT-MRI system S″ for simultaneously imaging, in accordance with an alternative embodiment of the present disclosure. When a CT component, e.g., the CT component 610, is substituted for a PET/CT component (not shown), two different patients (not shown) are simultaneously scanned, e.g., one patient is scanned by the CT component 610 while the other patient is scanned by an MR component, e.g., the MRI component 620, with a barrier, e.g., the movable barrier 640, being in a closed position. In the system S″, each of the MR component 620 and the CT component 610 has a corresponding transporter, e.g., a transporter 608a and a transporter 608b. Each corresponding transporter, e.g., the transporter 608a and the transporter 608b, is further configured to independently operate in relation to one another. The initial disposition of the transporter 608a is proximate the CT component 610 and opposite from the transporter 608b. The initial disposition of the transporter 608b is proximate the MR component 620 and opposite from the transporter 608a. By using the system S″, total imaging time is saved when a plurality of patients who need only one or two types of imaging are to be scanned in a day.

Still referring to FIG. 13, in this alternative embodiment, the MRI-CT system S″ comprises: a CT component 610, such as a CT scanner; an MR component 620, such as an MR scanner; transporters 608a, 608b; a bridge 630 configured to engage the transporters 608a, 608b and disposable in relation to the MR component 620; and a movable barrier 640, such as a removable barrier or door, disposable between the CT component 610 and the MR component 620. The movable barrier 640 comprises a magnetic shield. The CT component 610 and the MR component 620 are disposable in relation to one another in at least one of linearly aligned and colinearly aligned. The CT component 610, comprising the CT scanner, has a CT scanner limit of approximately 1 Gauss; however, for at least that the CT component 610 is operable with the movable barrier 640 comprising the magnetic shield and that the MR component 620 comprises an MR scanner having a small fringe field in a range of approximately 60 cm to approximately 300 cm for the distance of the 5-Gauss level as measured from a center of the MR component 620 in the absence of a magnetic shield, the MR component 620 is operable despite the CT scanner limit, thereby overcoming many challenges in the related art. The magnetic shield comprises at least one shield layer. The at least one shield layer comprises at least one magnetic shielding layer. The at least one magnetic shielding layer comprises a magnetic shielding material. The magnetic shielding material comprises a ferromagnetic material. The ferromagnetic material comprises at least one of silicon steel and any other iron alloy.

Still referring to FIG. 13, in an embodiment of the MRI-CT system S″, the movable barrier 640 comprises a RF shielding feature, such as an RF shield, the movable barrier 640 operating, at least in part, to protect the MR component 620 against RF noise emanating from the CT component 610 via the RF shielding feature. In some embodiments of the system S″, the bridge 630 comprises a plurality of portions, such as a first portion 631, a second portion 632, and a third portion 633, wherein each portion comprises at least one waveguide configured to at least one of respectively couple with at least one cable of the transporters 608a, 608b and respectively accommodate at least one least one cable of the transporters 608a, 608b. The bridge 630 is configured to engage the transporter 608a and to facilitate coupling the transporter 608a with the CT component 610 by way of at least one CT component coupler (not shown), such as a latching mechanism, e.g., disposed proximate, or within, the CT component 610. The bridge 630 is further configured to engage the transporter 608b and to facilitate coupling the transporter 608b with the MR component 620 by way of at least one MR component coupler (not shown), such as a latching mechanism, e.g., disposed proximate, or within, the MR component 620. The second portion 632 facilitates moving the transporter 608a from the CT component 620 to the MR component 620 and moving the transporter 608b from the MR component 620 to the CT component 610.

Still referring to FIG. 13, the movable barrier 640 further comprises at least one of at least one window (not shown) and at least one waveguide (not shown). The RF shielding feature protects the MR component 620 against RF noise emanating from the CT component 610 during MRI imaging. For most implementations of the system S″, the movable barrier 640 is disposed in an open position during MRI scanning by the MR component 620 and in a closed position during CT scanning by the CT component 610; however, for other implementations of the system S″, the movable barrier 640 is disposed either an open position or a closed position during MRI scanning by the MR component 620 and in a closed position during CT scanning by the CT component 610. The at least one shield layer further comprises at least one RF shield layer. The at least one RF shield layer comprises an RF shielding material. The RF shielding material comprises copper.

Referring back to FIGS. 6-13, advantages of the embodiments of the present disclosure include, but are not limited to: increased efficiency for imaging a subject that requires a plurality of serial imaging modalities; eliminating the need to transfer a subject in undue distances from imaging equipment of one imaging modality to another imaging equipment of another imaging modality; securing a subject's head in a same manner during both scanning procedures, thereby improving image co-registration; a subject passes through a CT scanner to reach an MR scanner, thereby shortening travel distance as well as travel time; a movable barrier or removable barrier, e.g., a door, prevents unauthorized access to MRI equipment; a movable barrier or removable barrier comprises a radiation shield to protect MRI equipment from CT ionizing radiation; a movable barrier or removable barrier comprises a magnetic shield to protect CT equipment from magnetic fields; a movable barrier or removable barrier comprises sliding doors, such as opening from left to right relative to the floor, a garage-style door, and a swing-style door, by examples only; a CT scan is used to determine whether a subject is suitable for MRI, e.g., conditions involving a piece of metal in a subject's eye, a pacemaker, etc.; easy retrofit of an existing, or manufacture of a new, CT table comprising carbon fiber, by fabricating a head section comprising fiberglass, rather than carbon fiber, thereby facilitating use of the table that is compatible for both CT and MR imaging modalities; optionally, with a fast-ramping magnet, the MRI equipment can be deactivated (or ramped to a lower magnetic field) during CT imaging; during the CT scan, RF coils (or components thereof) are removable, thereby reducing likelihood of image artifacts introduced in CT images due to the presence of metal and other discrete electronic components; beneficial facilitation for use of a head fixation cradle having RF coil fitted in relation to a subject's head that remains in both the CT scan and the MR scan scans; a PET detector ring array is integrable into the MR component, wherein the CT data provides attenuation correction data for the PET image; alternatively, the CT component is substitutable for a PET/CT component as CT scans are typically very short in duration, while MR scans and PET scans are longer in duration relative to CT scans, wherein greater total time savings is provided by simultaneously acquiring CT scans and MRI scans; each scan of a CT scan and an MRI scan are performable without performing the other scan if both scans are not required; and the system of the present disclosure comprises an MR component having small fringe field and a movable barrier having a magnetic shield, thereby overcomes the related art challenge of a CT scanner having a CT scanner limit of approximately 1 Gauss which, otherwise, would require that a CT scanner is located a great distance, e.g., in separate rooms, from an MR scanner.

Still referring back to FIGS. 6-13, advantages of the embodiments of the present disclosure include, but are not limited to: a co-location of a CT scanner and an MRI scanner offers advantages; providing a “one-stop shop” for head-imaging; eliminating the related art need to move a patient in relation to different imaging modalities; “assembly-line” configuration for transporting the patient through a CT scanner to reach an MRI scanner; and a CT scan initially determines whether a patient is suitable for an MRI scan, e.g., by identifying conditions, such as metal in the eye, a pacemaker, etc.

Information, as herein shown and described in detail, is fully capable of attaining the above-described embodiments of the present disclosure, the presently preferred embodiment of the present disclosure, and is, thus, representative of the subject matter which is broadly contemplated by the present disclosure. The scope of the present disclosure fully encompasses other embodiments and is to be limited, accordingly, by nothing other than the appended claims, wherein any reference to an element being made in the singular is not intended to mean “one and only one” unless explicitly so stated, but rather “one or more.” All structural and functional equivalents to the elements of the above-described preferred embodiment and additional embodiments are hereby expressly incorporated by reference and are intended to be encompassed as well by the present claims.

Moreover, no requirement exists for a system, apparatus, device, product-by-process, or method to address each and every problem sought to be resolved by the present disclosure, for such to be encompassed by the present claims. Furthermore, no element, component, or method step in the present disclosure is intended to be dedicated to the public regardless of whether the element, component, or method step is explicitly recited in the claims. However, that various changes and modifications in form, material, work-piece, and fabrication material detail may be made, without departing from the spirit and scope of the present disclosure, as set forth in the appended claims, are also encompassed by the present disclosure.

What is claimed:

1. An MRI-CT system for sequentially imaging a subject, the system comprising:
a CT component for initially performing CT imaging;
an MR component for subsequently performing MR imaging, the MR component and the CT component disposable in relation to one another in at least one of linearly aligned and colinearly aligned; and
a movable barrier disposable between the CT component and the MR component,
the movable barrier comprising a magnetic shield, and
the movable barrier disposable in one of an open position and a closed position during MRI scanning by the MR component and in a closed position during CT scanning by the CT component.

2. The system of claim 1, further comprising a bridge having at least one portion,
the bridge configured to engage a transporter, and
the bridge and disposable between the CT component and the MR component.

3. The system of claim 2, further comprising the transporter, the transporter configured to engage the bridge.

4. The system of claim 1,
wherein the CT component comprises a CT scanner having a CT scanner limit of approximately 1 Gauss,
wherein the MR component comprises an MR scanner having a small fringe field in a range of approximately 60 cm to approximately 300 cm for the distance of the 5-Gauss level as measured from a center of the MR component in the absence of a magnetic shield, and
whereby the MR component is operable regardless of the CT scanner limit.

5. The system of claim 1,
wherein the movable barrier comprises at least one of at least one removable barrier, at least one door, at least one window, and at least one waveguide,
wherein the movable barrier further comprises a radio-frequency (RF) shield, and
whereby the movable barrier, via the RF shield, operating, at least in part, protects the MR component against RF noise emanating from the CT component during MRI imaging.

6. The system of claim 1, wherein the bridge is further configured to facilitate coupling the transporter with the MR component by way of at least one MR component coupler.

7. The system of claim 2, wherein the at least one portion comprises a plurality of portions, wherein each at least one portion comprises at least one waveguide configured to at least one of:
couple with at least one cable of the transporter; and
accommodate at least one cable of the transporter.

8. The system of claim 2,
wherein the plurality of portions comprises a first portion and a second portion,
wherein the first portion is configured to firstly move the transporter to, and through, the CT component when the transporter is disposed in relation to the first portion, and
wherein the second portion is configured to secondly move the transporter 608 to, and, at least partially, into, the MR component when the transporter is disposed is disposed in relation to the second portion.

9. The system of claim 3, wherein at least one of the bridge and the transporter comprises a moving feature to facilitate moving the transporter forward in a sequence and backward from the sequence.

10. The system of claim 3,
wherein the transporter comprises a material being compatible with CT scanning requirements of the CT component and MR scanning requirements of the MR component,
wherein the transporter comprises a first section and a section,
wherein the first section corresponds to a location for accommodating a subject's head and shoulders are disposed,
wherein the second section corresponds to a location for accommodating a subject's torso,
wherein the first section comprises fiberglass,
wherein the first section is configured for disposition in the MR component during operation of the system in an MR imaging mode.

11. A method of providing an MRI-CT system for sequentially imaging a subject, the method comprising:
providing a CT component for initially performing CT imaging;
providing an MR component for subsequently performing MR imaging, the MR component and the CT component disposable in relation to one another in at least one of linearly aligned and colinearly aligned; and
providing a movable barrier disposable between the CT component and the MR component,
providing the movable barrier comprising providing a magnetic shield, and
providing the movable barrier comprising providing the movable barrier as disposable in one of an open position and a closed position during MRI scanning by the MR component and in a closed position during CT scanning by the CT component.

12. The method of claim 11, further comprising providing a bridge having at least one portion,
providing the bridge comprising configuring the bridge to engage a transporter, and
providing the bridge comprising configuring the bridge as disposable between the CT component and the MR component.

13. The method of claim 12, further comprising providing the transporter, providing the transporter comprising configuring the transporter to engage the bridge, wherein at least one of providing the bridge and providing the transporter comprises providing a moving feature to facilitate moving the transporter forward in a sequence and backward from the sequence.

14. The method of claim 11,
wherein providing the CT component comprises providing a CT scanner having a CT scanner limit of approximately 1 Gauss,
wherein providing the MR component comprises providing an MR scanner having a small fringe field in a range of approximately 60 cm to approximately 300 cm for the distance of the 5-Gauss level as measured from a center of the MR component in the absence of a magnetic shield, and
whereby the MR component is operable regardless of the CT scanner limit.

15. The method of claim 11,
wherein providing the movable barrier comprises providing at least one of at least one removable barrier, at least one door, at least one window, and at least one waveguide,
wherein providing the movable barrier further comprises providing a radio-frequency (RF) shield, and
whereby the movable barrier, via the RF shield, operating, at least in part, protects the MR component against RF noise emanating from the CT component during MRI imaging.

16. The method of claim 11, wherein providing the bridge further comprises configuring the bridge to facilitate coupling the transporter with the MR component by way of at least one MR component coupler.

17. The method of claim 12, wherein providing the bridge having the at least one portion comprises providing a plurality of portions, wherein providing the plurality of portions comprises providing each portion of the plurality of portions with at least one waveguide configured to at least one of:
couple with at least one cable of the transporter; and
accommodate at least one cable of the transporter.

18. The method of claim 12,
wherein providing the bridge having the at least one portion comprises providing a plurality of portions,
wherein providing the plurality of portions comprises providing a first portion and providing a second portion,
wherein providing the first portion comprises configuring the first portion to firstly move the transporter to, and through, the CT component when the transporter is disposed in relation to the first portion, and
wherein providing the second portion comprises configuring the second portion to secondly move the transporter to, and, at least partially, into, the MR component when the transporter is disposed is disposed in relation to the second portion.

19. The method of claim 13,
wherein providing the transporter comprises providing a material being compatible with CT scanning requirements of the CT component and MR scanning requirements of the MR component,
wherein providing the transporter comprises providing a first section and providing a second section,
wherein providing the first section comprises providing the first section corresponding to a location for accommodating a head and shoulders of the subject,
wherein providing the second section comprises providing the second section corresponding to a location for accommodating a torso of the subject,
wherein providing the first section comprises providing fiberglass, and
wherein providing the first section comprises configuring the first section for disposition in the MR component during operation of the system in an MR imaging mode.

20. A method of sequentially imaging a subject by way of an MRI-CT system, the system comprising:
providing the MRI-CT system, providing the MRI-CT system comprising: providing a CT component for initially performing CT imaging; providing an MR component for subsequently performing MR imaging, the MR component and the CT component disposable in relation to one another in at least one of linearly aligned and colinearly aligned; and providing a movable barrier disposable between the CT component and the MR component, providing the movable barrier comprising providing a magnetic shield, and providing the movable barrier comprising providing the movable barrier as disposable in one of an open position and a closed position during MRI scanning by the MR component and in a closed position during CT scanning by the CT component;
providing a bridge having at least one portion, providing the bridge comprising configuring the bridge to engage a transporter, and providing the bridge comprising configuring the bridge as disposable between the CT component and the MR component;
providing the transporter, providing the transporter comprising configuring the transporter to engage the bridge, wherein at least one of providing the bridge and providing the transporter comprises providing a moving feature to facilitate moving the transporter forward in a sequence and backward from the sequence;
disposing the subject on the transporter;
engaging the transporter with the bridge;
moving the transporter in relation to the CT component by using the bridge;
performing CT imaging of the subject by using the CT component;
moving the transporter in relation to the MR component by using the bridge; and
performing MR imaging of the subject by using the MR component.
